(19) United States
(12) Patent Application Publication
(10) Pub. No.: US 2020/0068846 A1
(43) Pub. Date: Mar. 5, 2020

(54) NATURE NET

(71) Applicant: Paula J. Rolf, Hopkins, MN (US)

(72) Inventor: Paula J. Rolf, Hopkins, MN (US)

(21) Appl. No.: 16/559,441

(22) Filed: Sep. 3, 2019

Related U.S. Application Data
(60) Provisional application No. 62/725,896, filed on Aug. 31, 2018.

(51) Int. Cl. A01K 1/035; A01K 67/033; A01K 31/12; A01K 63/00; A01G 13/04; A01K 29/00; A01K 31/08

(52) U.S. Cl. CPC A01K 1/035; A01K 67/033; A01K 31/12; A01G 2013/006; A01G 13/043; A01K 29/005; A01K 31/08; A01K 63/003

(57) ABSTRACT

Various indoor/outdoor nature net configurations are disclosed herein. In some embodiments, an indoor/outdoor nature net enclosure may include an upper section, a lower section, and a middle section. The middle section may extend between the upper section and the lower section to define an interior space. The middle section may include a two-way zipper that extends from the upper section to the lower section. Some net enclosures disclosed herein may be used in connection with a wire frame. The wire frame may be configured to be positioned in the interior space and to support the net enclosure. In some embodiments, the wire frame may include a plurality of vertical members and a plurality of horizontal members.

### CROSS-REFERENCE TO RELATED APPLICATIONS

This application claims the benefit of U.S. Provisional Application No. 62/725,896, filed Aug. 31, 2018, the contents of which are incorporated herein by reference.

### TECHNICAL FIELD

This disclosure relates generally to indoor/outdoor nature nets.

### BACKGROUND

Netting may have a variety of uses in the outdoors. In many instances, relatively small net configurations may be used to house animals. For example, net enclosures may provide shelter for monarch butterflies, with milkweed in the interior and with chrysalises attaching to the inner surface of the net enclosure. Other animals (e.g., birds, bugs, spiders, salamanders, frogs, reptiles, pets, etc.) may be kept inside such net enclosures. Net enclosures may also be used to cover plants (e.g., vegetables, flowers, native plants, house plants, shrubs, trees, etc.). Netting can protect enclosed plants from insects and other environmental hazards. Such nets may be used in various indoor applications as well.

### SUMMARY

Net enclosure embodiments disclosed herein have various features for enabling users to accomplish several objectives, depending on the particular application and situation. Some embodiments have a two-way zipper that enables users to access the net enclosure interior space either from the top (e.g., to attend to something associated with a monarch chrysalis or with the top of a plant) or from the bottom (e.g., to attend to something with the ground). When accessing the interior space from the top, the bottom may remain closed, and vice versa.

Many net enclosure embodiments disclosed herein may be used in connection with an expandable/collapsible wire frame. The net enclosure may have apertures on the top and/or bottom, and vertical members of the wire frame may poke through the apertures to secure the wire frame to the ground and the net enclosure to the wire frame. Some net enclosure embodiments may be configured to cover a potted plant, and the lower edge of the net enclosure may be cinched around the side of the pot.

Some net enclosures may include an access door for easily accessing an upper portion of the interior space. The access door may be especially useful for releasing newly hatched monarch butterflies into the wild. In some embodiments the access door may be opened, folded under, and secured to the net enclosure to form a perch for an animal housed within the net enclosure. In some instances, net enclosures may be used for wildlife rescue and relocation with simple and gentle release through the access door (or two-way zipper opening).

Some net enclosures may be used to cover plants indoors. People with pets may be unable to keep plants indoors and may thus be deprived of the benefits associated with indoor plants. Net enclosures such as those discussed herein may be used to prevent pets from getting into plants that are kept indoors.

Inventive principles described herein may be incorporated into a variety of different indoor/outdoor nature net configurations. The details of one or more examples are set forth in the accompanying drawings and the description below. Other features, objects, and advantages will be apparent from the description and drawings.

### BRIEF DESCRIPTION OF DRAWINGS

The following drawings are illustrative of particular embodiments of the present invention and therefore do not limit the scope of the invention. The drawings are not necessarily to scale (unless so stated) and are intended for use in conjunction with the explanations in the following description. Embodiments of the invention will hereinafter be described in conjunction with the appended drawings, wherein like numerals denote like elements.

FIG. 1A is a perspective view of an illustrative indoor/outdoor nature net assembly with a two-way zipper in a closed position.

FIG. 1B is a perspective view of the indoor/outdoor nature net assembly of FIG. 1A with a two-way zipper being open in its upper part and closed in its lower part.

FIG. 1C is a perspective view of the indoor/outdoor nature net assembly of FIG. 1A with a two-way zipper being open in its upper part and closed in its lower part.

FIG. 2A is a closer perspective view of the indoor/outdoor nature net assembly of FIG. 1A with an access door in a closed position.

FIG. 2B is a closer perspective view of the indoor/outdoor nature net assembly of FIG. 1A with the access door in an open position.

FIG. 2C is a closer perspective view of the indoor/outdoor nature net assembly of FIG. 1A with the access door in a perched position.

FIG. 3A is a perspective view of an illustrative indoor/outdoor nature net enclosure with an upper section opening in a closed position.

FIG. 3B is a perspective view of the indoor/outdoor nature net enclosure of FIG. 3A with the upper section opening in an open position.

FIG. 4A is a perspective view of an illustrative indoor/outdoor nature net enclosure with upper and lower section openings both in a closed position.

FIG. 4B is a perspective view of the indoor/outdoor nature net enclosure of FIG. 4A with the upper and lower section openings both in an open position.

FIG. 5A is a perspective view of an illustrative indoor/outdoor nature net assembly with part of a cover assembly covering a net enclosure and wire frame.

FIG. 5B is a perspective view of the indoor/outdoor nature net assembly of FIG. 5A with the cover assembly covering the net enclosure and wire frame.

FIG. 6 is a perspective view of an illustrative indoor/outdoor nature net assembly with a cap covering an upper section of a net enclosure.

FIG. 7 shows an illustrative wire frame in a collapsed state.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

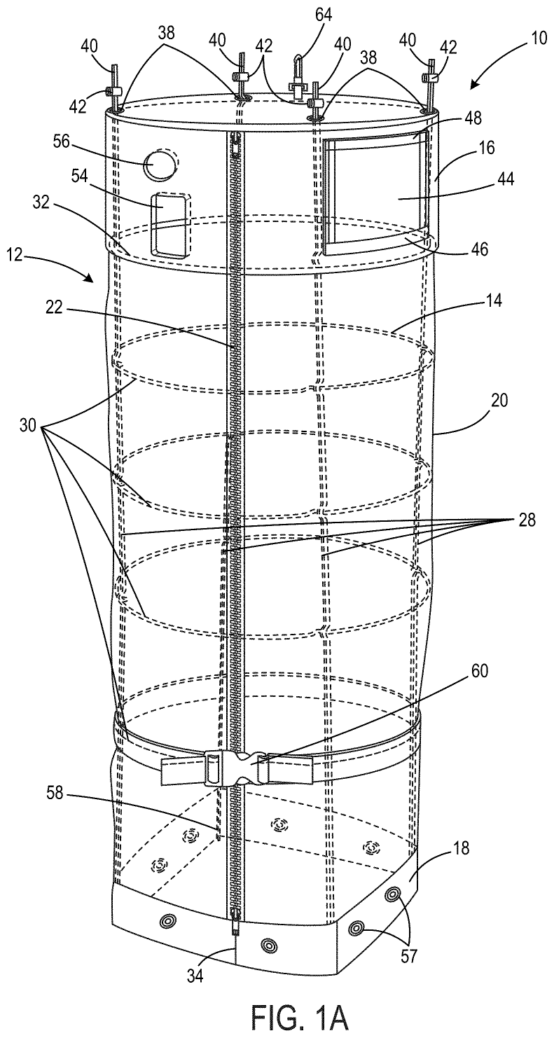

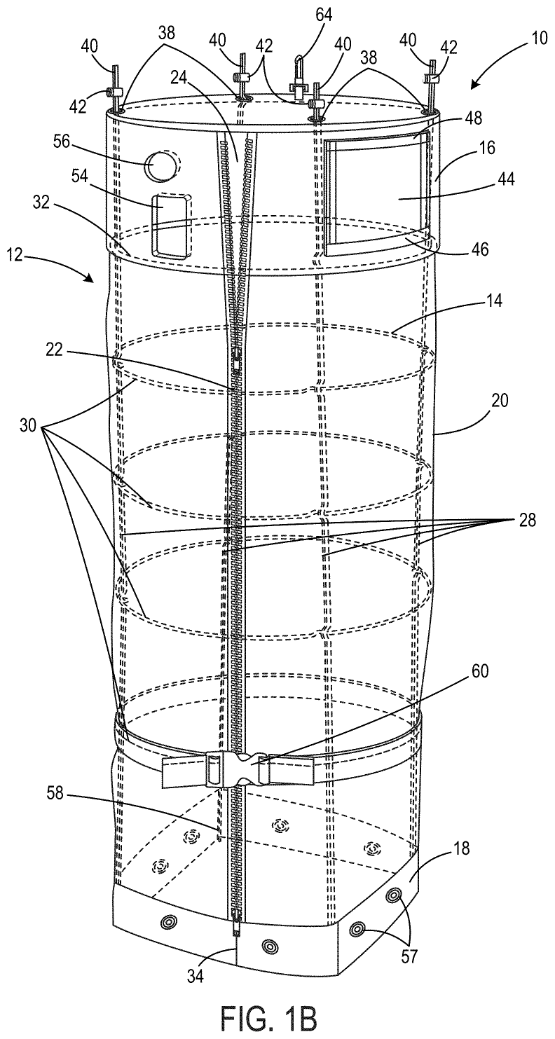

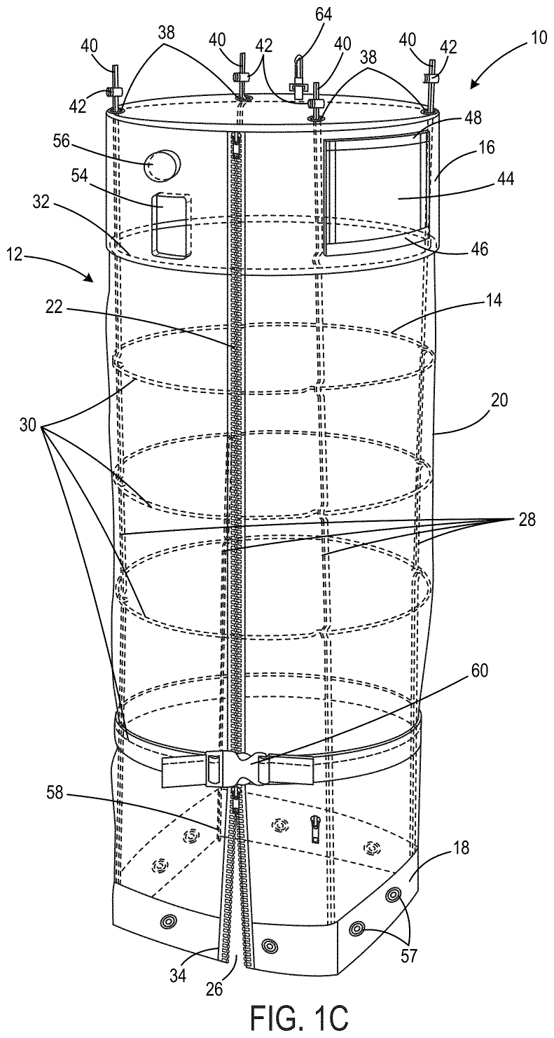

FIGS. 1A-1C show an indoor/outdoor nature net assembly 10 according to some embodiments of the present invention. As shown, the indoor/outdoor nature net assembly can include a net enclosure 12 and a wire frame 14. The net enclosure 12 of FIGS. 1A-1C may include an upper section 16, a lower section 18, and a middle section 20 that extends between the upper section 16 and the lower section 18 to define an interior space. As discussed elsewhere herein, a variety of things may be enclosed in the interior space.

In some embodiments, the middle section 20 of the net enclosure 12 includes a two-way zipper 22 that extends from the upper section 16 to the lower section 18 of the net enclosure 12. The two-way zipper 22 may be a full-length zipper. As shown, the two-way zipper 22 has an upper part 24 and a lower part 26. The two-way zipper 22 may be configured to permit access to the interior space defined by the net enclosure 12 from the upper section 16 of the net enclosure 12 while the lower part 26 of the two-way zipper 22 remains closed (FIG. 1B). The two-way zipper 22 may be configured to also permit access to the interior space defined by the net enclosure 12 from the lower section 18 of the net enclosure 12 while the upper part 24 of the two-way zipper 22 remains closed (FIG. 1C). Such a two-way zipper 22 can facilitate easy access to any part of the interior space in a convenient manner.

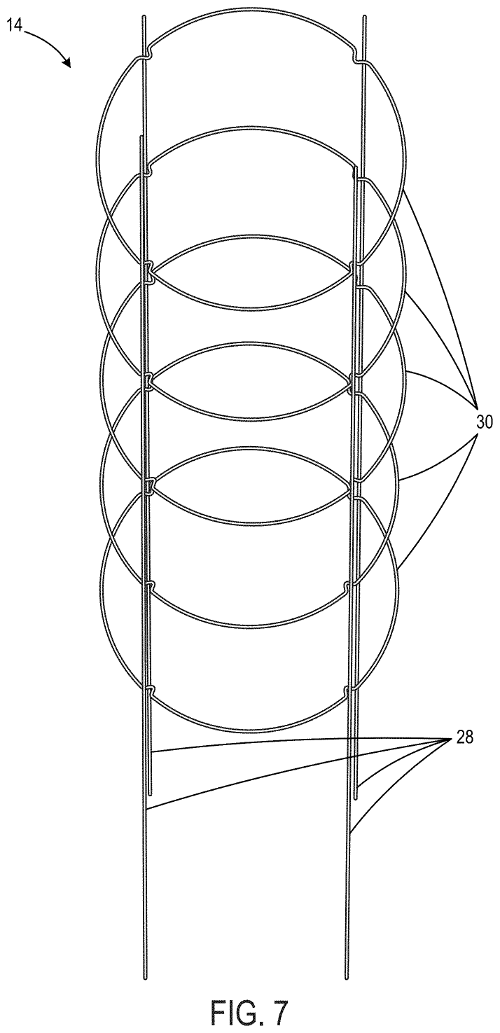

In many embodiments, the wire frame 14 may be configured to be positioned in the interior space and to support the net enclosure 12. The wire frame 14 may include a plurality of vertical members 28 and a plurality of horizontal members 30. FIGS. 1A-1C show the wire frame 14 in an expanded state. FIG. 7 shows the wire frame 14 in a collapsed state. In installing the net enclosure 12 over the wire frame 14, the two-way zipper 22 may be opened in its lower part 26 to allow the upper section 16 to be positioned over the wire frame 14. The lower part 26 of the two-way zipper 22 may then be zipped closed to enclose the interior space.

Different portions of the net enclosure 12 may have different thicknesses. For example, in some embodiments, the middle section 20 of the net enclosure 12 may be made of a first material, and the upper section 16 may be made of a second material that is thicker than the first material. In some such embodiments, the upper section 16 may extend down along a side of the wire frame 14 below the uppermost horizontal member 32 of the wire frame 14 in order to protect against an animal being pinched between the lower end of the upper section 16 and the uppermost horizontal member 32. In many such embodiments, the upper section 16 may extend around a perimeter of the wire frame 14. In many instances, an upper section 16 that is made of a thicker material than that of the middle section 20 may enable the upper section 16 to protect against rain or other elements driving into the top of the interior space. In some embodiments, the middle section 20 of the net enclosure 12 may be made of a first material, and the lower section 18 may be made of a second material that is thicker than the first material. In some such embodiments, the lower section 18 may include a water-resistant closure 34 configured to close over where the two-way zipper 22 interfaces with a lower perimeter of the lower section 18.

As noted, the net enclosure 12 may be constructed of a variety of materials depending on the application. For example, in embodiments in which the upper section 16 is made of a material that is thicker than the material of the middle section 20, the upper section 16 may be made of canvas. A canvas upper section 16 can provide an especially good surface for monarch chrysalis attachment. In some embodiments, the upper section 16 and the middle section 20 may be made of a mesh material. In some such embodiments, the upper section 16 may include an extra layer of the mesh material than the middle section 20. Some such embodiments may serve as shelter for various kinds of animals. In some embodiments, the net enclosure 12 may be made of heavy duty UV protected netting. Such embodiments may protect the interior space from direct sunlight. In some embodiments, some or all of the net enclosure 12 may be made of a breathable shower curtain material to provide water resistance. In some embodiments, the net enclosure 12 may be made of cotton.

FIGS. 1A-1C show an illustrative way in which the upper section 16 of the net enclosure 12 may interface with the wire frame 14. The upper section 16 may include at least one upper section aperture 38 configured to receive an upper end 40 of at least one of the vertical members 28 of the wire frame 14. In some embodiments, at least one lock 42 may be configured to attach to the vertical member(s) 28 to prevent the upper section aperture(s) 38 from inadvertently slipping off the vertical member(s) 28. FIGS. 1A-1C show four vertical members 28, four upper section apertures 38, and four locks 42, but other embodiments may include any suitable number of vertical members 28, upper section apertures 37, and locks 42.

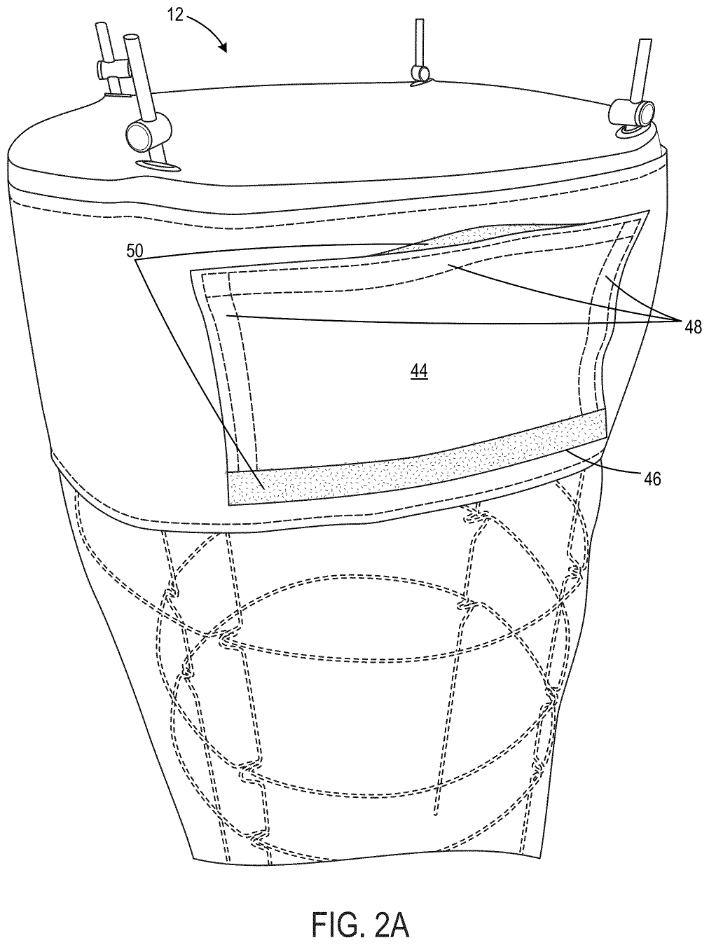

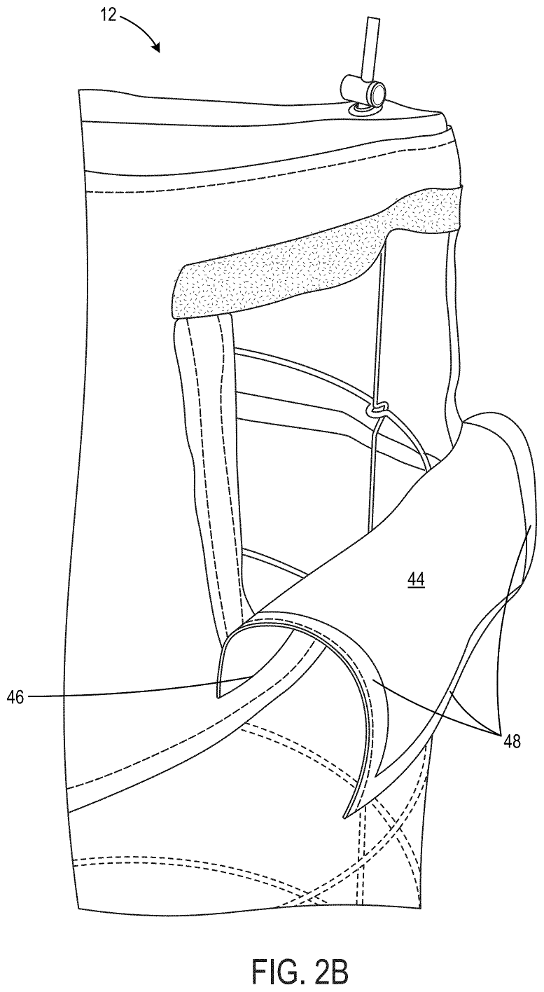

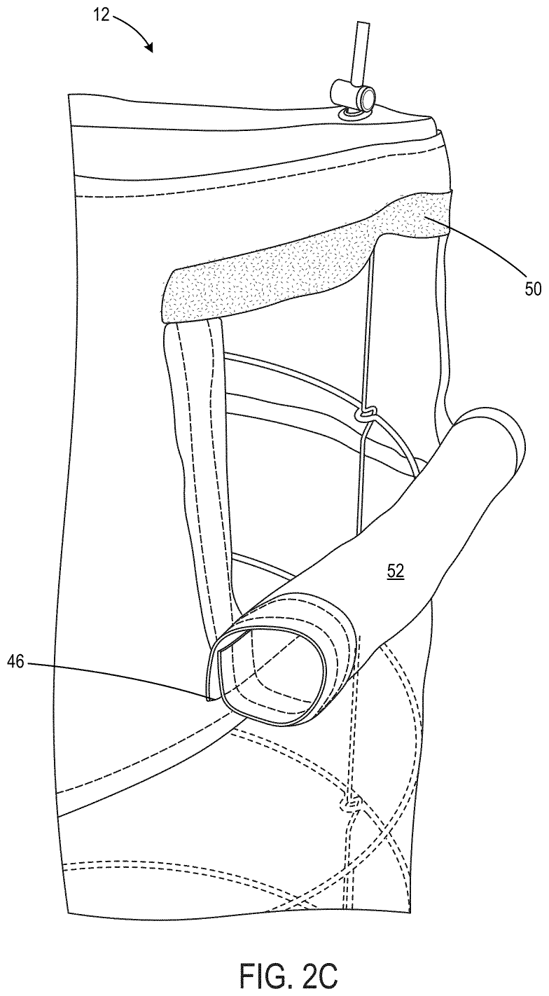

As shown in FIGS. 1A-1C, the net enclosure 12 may include an access door 44 that permits access to the interior space defined by the net enclosure 12. FIGS. 2A-2C show the access door 44 and the net enclosure 12 in greater detail. The access door 44 may be hinged along its lower edge 46. In some embodiments, the access door 44 may be releasably fastened to the upper section 16 of the net enclosure 12 along at least one non-hinged edge 48 of the access door 44 via a hook-and-loop fastener 50. As shown, the access door 44 is rectangular in shape, but various shapes of access doors 44 may be suitable in different applications. FIG. 2A shows the access door 44 in a closed position. FIG. 2B shows the access door 44 in an open position. FIG. 2C shows how the non-hinged edge 48 of the access door 44 may be releasably fastened to the lower edge 46 of the access door 44 via the hook-and-loop fastener 50 when in an open position to create a perch 52. Such an access door 44 provides easy access to the interior space without having to use the two-way zipper 22. This can be especially useful for releasing newly hatched monarch butterflies into the wild and/or for enabling animals to climb onto the perch 52.

Referring again to FIGS. 1A-1C, the net enclosure 12 may include features that permit viewing the interior space defined by the net enclosure 12. For example, the upper section 16 of the net enclosure 12 may include a camera mount 54 configured to facilitate imaging of the interior space. In another example, the upper section 16 of the net enclosure may include a viewing window 56 made of a material that is more transparent than other portions of the upper section 16. As shown, a mobile device may be positioned in the camera mount 54, and a lens of the mobile device's camera may be aligned with the viewing window 56. In situations in which no mobile device is positioned in the camera mount 54, a user may look into the interior space through the viewing window 56.

The lower section 18 of the net enclosure 12 may be anchored when in use. For example, in some embodiments, the lower section 18 may include at least one lower section aperture 57 configured to receive the lower end 58 of at least one of the vertical members 28 of the wire frame 14 for staking the lower section 18 to the ground. In many embodiments, separate stakes may be driven through the lower section apertures 57 into the ground to anchor the lower section 18 of the net enclosure 12. Eight lower section apertures 57 are shown, but other embodiments may include any suitable number of lower section apertures 57. In some embodiments, the lower section 18 may include an elongate fastener 60 configured to permit adjustment of a lower section opening for securing the lower section 18 to a pot. Including both kinds of anchoring structures in the same net enclosure 12 may increase the versatility of the net enclosure 12. When the lower section 18 of the net enclosure 12 is anchored in use, unwanted animals and/or environmental elements may be inhibited from entering the interior space.

In some embodiments, the net enclosure 12 may include a hook 64 on an exterior surface of the upper section 16. The hook 64 may be positioned on an upper perimeter of the upper section 16 opposite where the two-way zipper 22 interfaces with the upper perimeter of the upper section 16. The hook 64 may be used for longer-term storage and/or for shorter-term drying/cleaning.

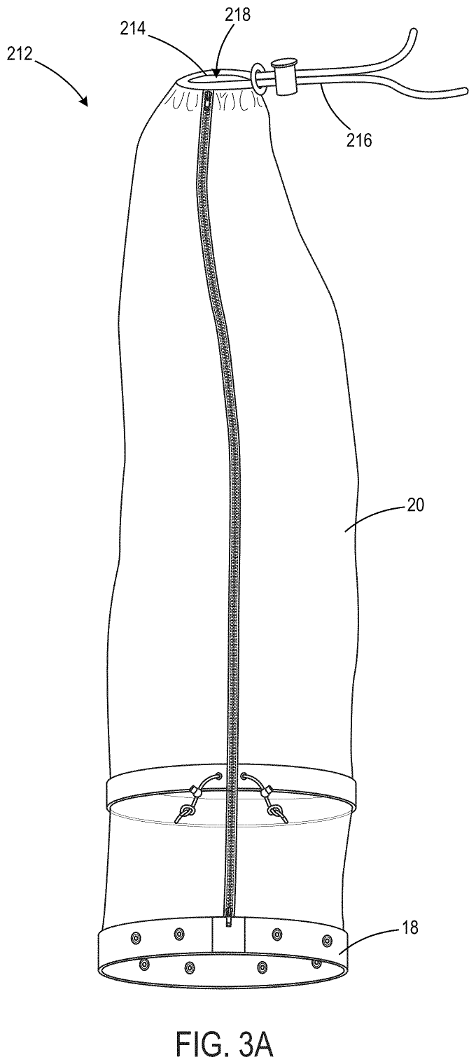

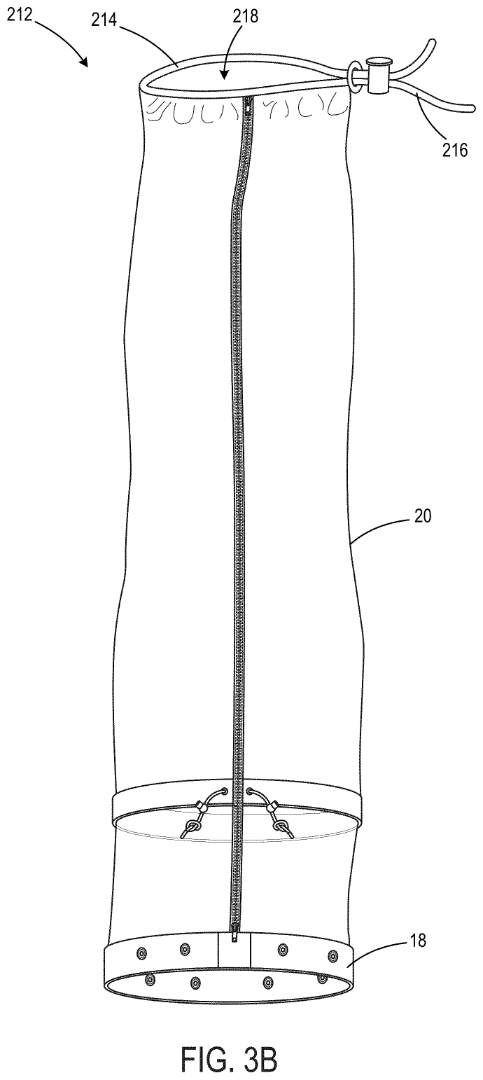

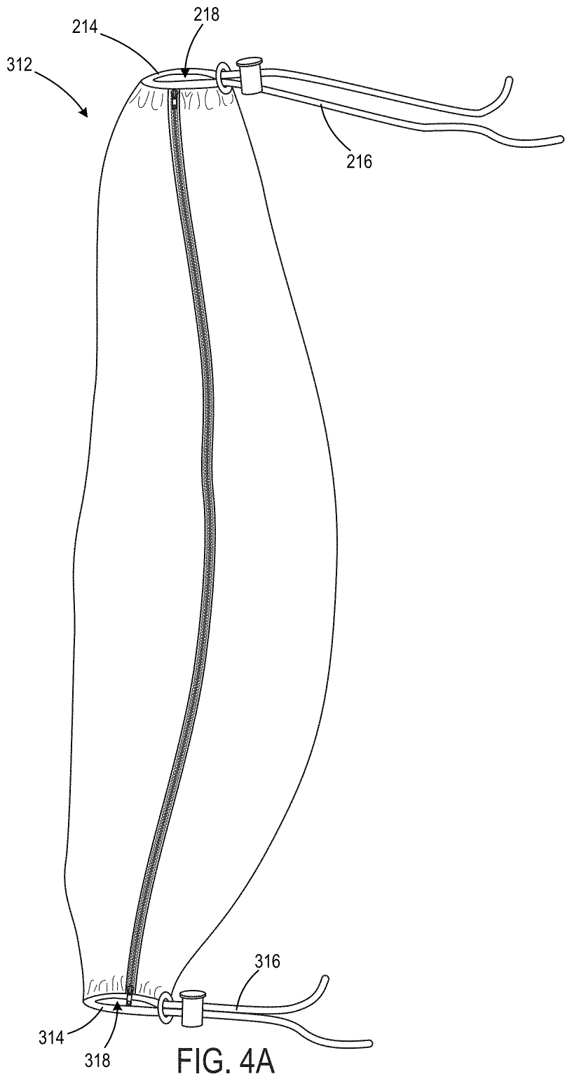

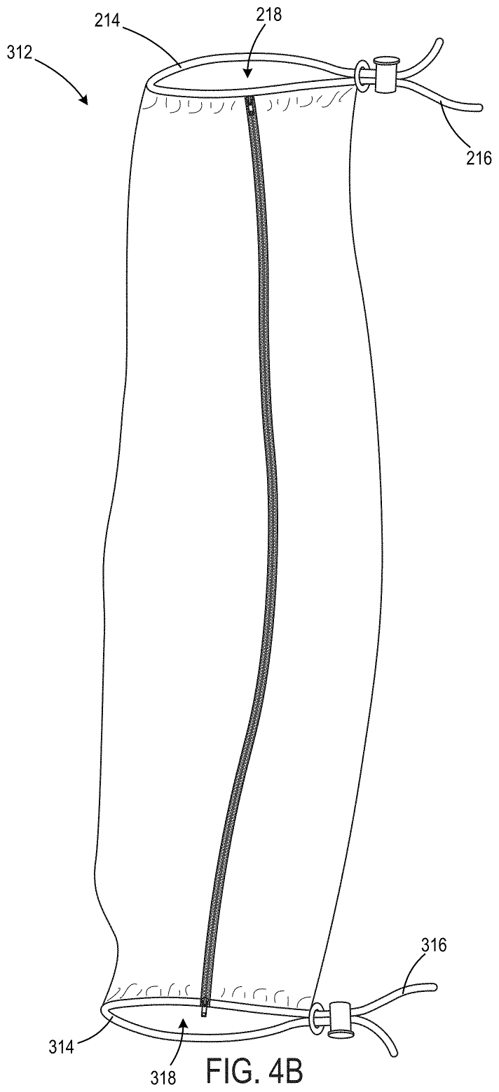

FIGS. 3A-3B and 4A-4B show net enclosures 212, 312 with an upper section 214 that differs from the upper section 214 of FIGS. 1A-1C. Such net enclosures 212, 312 may be used in connection with a wire frame or without a wire frame. Referring to FIGS. 3A-3B and 4A-4B, the upper section 214 of the net enclosures 212, 312 may include a first elongate fastener 216. The first elongate fastener 216 may be configured to permit adjustment of an upper section opening 218 between a closed position and an open position. FIGS. 3A and 4A show the upper section opening 218 in the closed position. FIGS. 3B and 4B show the upper section opening 218 in the open position, with the upper section opening 218 having a cross-sectional area that equals a cross-sectional area of the middle section 20. When the upper section opening 218 is in the closed position, unwanted animals and/or environmental elements may be inhibited from entering the interior space through the upper section 214. When the upper section opening 218 is in the open position, the net enclosure 214 may be temporarily lowered to permit access to what the net enclosure 214 was enclosing. After that temporary period, the net enclosure 12 may again be raised, and the upper section opening 218 may again be closed.

The net enclosure 212 of FIGS. 3A-3B includes a middle section 20 and lower section 18 similar to those of the net enclosure 12 of FIGS. 1A-1C. The middle section 20 and lower section 18 may have similar characteristics to net enclosures 12 discussed herein and may be made from materials similar to those discussed herein. The lower section 18 of the net enclosure 212 may be anchored in a manner similar to anchoring techniques discussed herein.

The net enclosure 312 of FIGS. 4A-4B includes a lower section 314 that may be implemented in some embodiments. The lower section 314 may include a second elongate fastener 316. The second elongate fastener 316 may be configured to permit adjustment of a lower section opening 318 between a closed position and an open position. FIG. 4A shows the lower section opening 318 in the closed position. FIG. 4B shows the lower section opening 318 in the open position, with the lower section opening 318 having a cross-sectional area that equals the cross-sectional area of the middle section 20. In some embodiments, the lower section opening 318 may be tightened around a plant when in the closed position. Such a net enclosure 312 may be easily installed over a plant and/or a frame for purposes of defining an interior space.

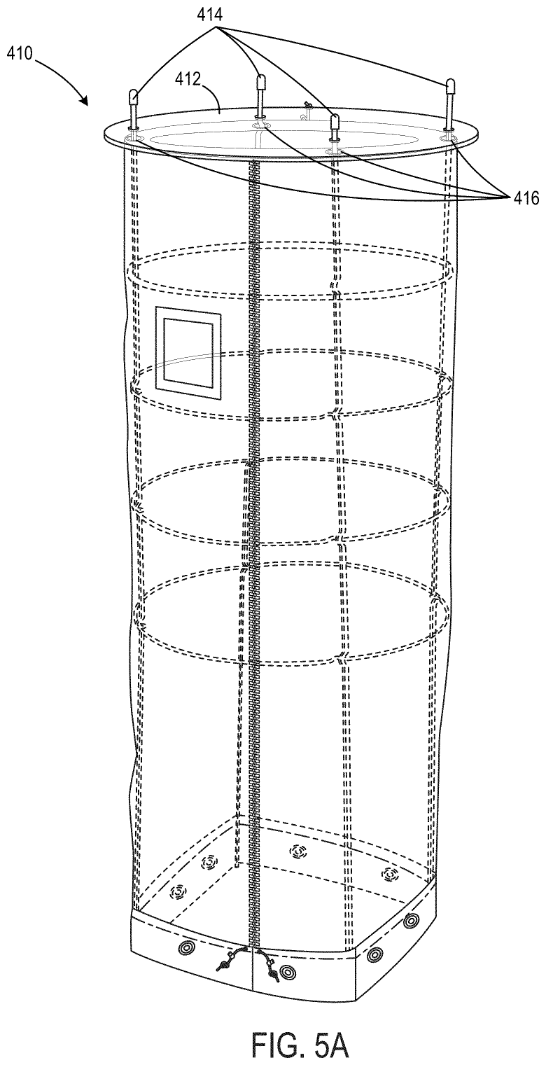

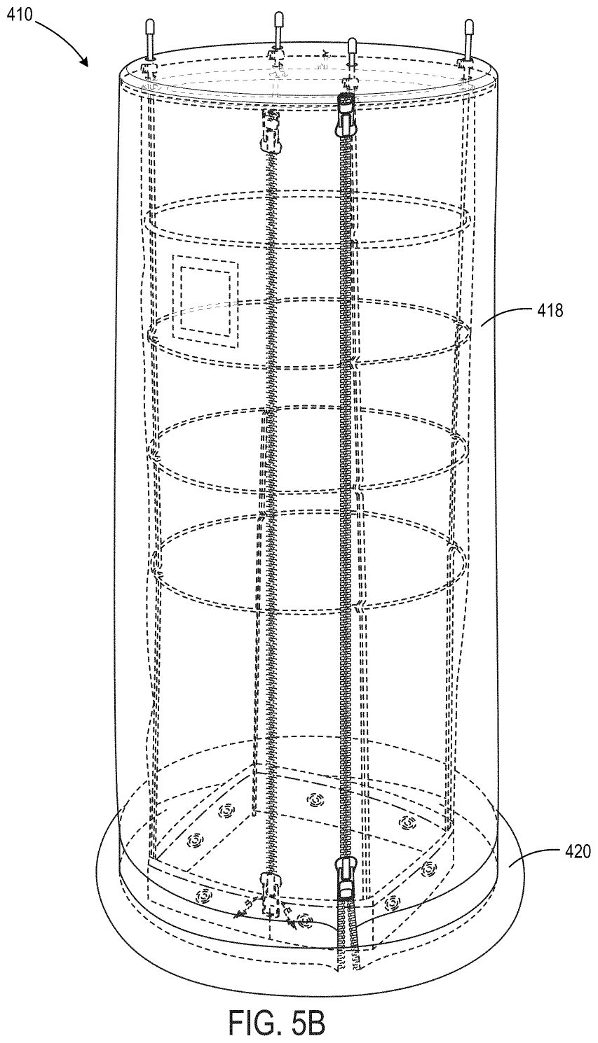

FIGS. 5A-5B shows a cover assembly 410 that can be used to cover a net enclosure and wire frame in some embodiments. Referring to FIG. 5A, the cover assembly 410 can include a plate 412 that interfaces with the upper end 414 of the wire frame 14. The plate 412 can include a plate aperture 416 configured to receive the upper end 414 of at least one of the vertical members 28 of the wire frame 14. As shown, the plate 412 includes four plate apertures 416 receiving the upper ends 414 of four vertical members of the wire frame, but other embodiments may include any suitable number of plate apertures 416, upper ends 414, and vertical members. Referring to FIG. 5B, in some embodiments, the cover assembly 410 can include a cover 418 configured to enclose the net enclosure and the wire frame. The cover 418 may be made of a heavier material (e.g., canvas) to provide a layer of insulation over the net enclosure 12 and the interior space. The cover 418 may be configured to work with net enclosures and wire frames such as those discussed herein.

The cover assembly 410 of FIGS. 5A-5B may include a sock 420 configured to be filled with sand and wrapped around a lower perimeter of the lower section 18 of the net enclosure 12 (see FIG. 5B). The sock 420 may be used in connection with other net enclosures discussed herein, including the net enclosure 12 of FIGS. 1A-1C.

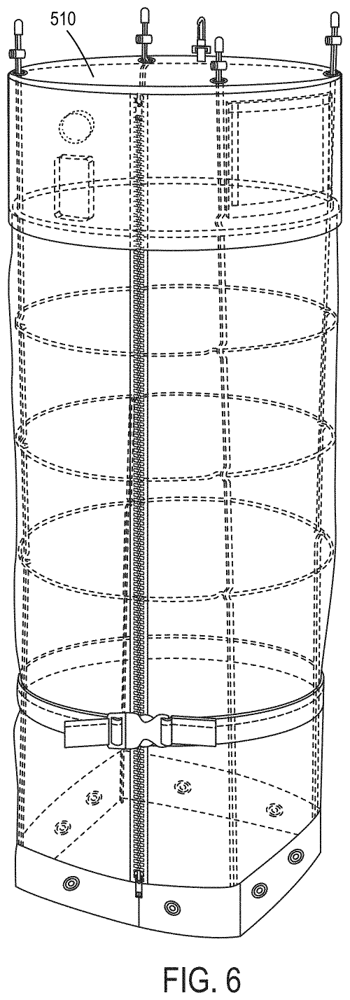

FIG. 6 shows a cap 510 that can be used in connection with a net enclosure and/or wire frame like those discussed herein. In some embodiments, the cap 510 may be configured to cover the upper section of the net enclosure. The cap 510 may be made of water-resistant material to protect the net enclosure 12 and the interior space from water, especially of driving rain that may cause damage to what is enclosed in the interior space.

Various examples have been described with reference to certain disclosed embodiments. The embodiments are presented for purposes of illustration and not limitation. One skilled in the art will appreciate that various changes, adaptations, and modifications can be made without departing from the scope of the invention.

What is claimed is:

1. An indoor/outdoor nature net enclosure comprising:
an upper section;
a lower section; and
a middle section extending between the upper section and the lower section to define an interior space, the middle section including a two-way zipper that extends from the upper section to the lower section, the two-way zipper having an upper part and a lower part, the two-way zipper being configured to permit access to the interior space from the upper section while the lower part of the two-way zipper remains closed and from the lower section while the upper part of the two-way zipper remains closed.

2. The indoor/outdoor nature net enclosure of claim 1, wherein the upper section, the lower section, and the middle section are configured to be supported by a wire frame in the interior space that includes a plurality of vertical members and a plurality of horizontal members.

3. The indoor/outdoor nature net enclosure of claim 2, wherein the middle section is made of a first material, and the upper section is made of a second material that is thicker than the first material, the upper section extending (a) down along a side of the wire frame below an uppermost horizontal member of the plurality of horizontal members of the wire frame and (b) around a perimeter of the wire frame.

4. The indoor/outdoor nature net enclosure of claim 3, wherein the upper section includes an access door hinged along a lower edge of the access door.

5. The indoor/outdoor nature net enclosure of claim 4, wherein the access door is releasably fastened to the upper section along at least one non-hinged edge of the access door via a hook-and-loop fastener when in a closed position.

6. The indoor/outdoor nature net enclosure of claim 5, wherein the at least one non-hinged edge of the access door is releasably fastened to the lower edge of the access door via the hook-and-loop fastener when in an open position to create a perch.

7. The indoor/outdoor nature net enclosure of claim 3, wherein the second material comprises canvas.

8. The indoor/outdoor nature net enclosure of claim 3, wherein the first material and the second material each comprise a mesh material, with the second material including an extra layer of the mesh material.

9. The indoor/outdoor nature net enclosure of claim 2, wherein the upper section includes at least one upper section aperture configured to receive an upper end of at least one of the vertical members of the wire frame.

10. The indoor/outdoor nature net enclosure of claim 2, wherein the upper section comprises a camera mount configured to facilitate imaging of the interior space.

11. The indoor/outdoor nature net enclosure of claim 2, wherein the upper section comprises a viewing window made of a material that is more transparent than other portions of the upper section.

12. The indoor/outdoor nature net enclosure of claim 2, wherein the lower section includes at least one lower section aperture configured to receive a lower end of at least one of the vertical members of the wire frame for staking the lower section to the ground, the lower section further including an elongate fastener configured to permit adjustment of a lower section opening for securing the lower section to a pot.

13. The indoor/outdoor nature net enclosure of claim 1, further comprising a hook on an exterior surface of the upper section, the hook being positioned on an upper perimeter of the upper section opposite where the two-way zipper interfaces with the upper perimeter of the upper section.

14. The indoor/outdoor nature net enclosure of claim 1, wherein the upper section includes a first elongate fastener configured to permit adjustment of an upper section opening between a closed position and an open position in which the upper section opening has a cross-sectional area that equals a cross-sectional area of the middle section.

15. The indoor/outdoor nature net enclosure of claim 14, wherein the lower section includes a second elongate fastener configured to permit adjustment of a lower section opening between a closed position and an open position in which the lower section opening has a cross-sectional area that equals the cross-sectional area of the middle section.

16. The indoor/outdoor nature net enclosure of claim 1, wherein the middle section is made of a first material, and the lower section is made of a second material that is thicker than the first material.

17. The indoor/outdoor nature net enclosure of claim 16, wherein the lower section includes a water-resistant closure configured to close over where the two-way zipper interfaces with a lower perimeter of the lower section.

18. An indoor/outdoor nature net assembly comprising:
a net enclosure that includes:
an upper section,
a lower section, and
a middle section extending between the upper section and the lower section to define an interior space, the middle section including a two-way zipper that extends from the upper section to the lower section, the two-way zipper having an upper part and a lower part, the two-way zipper being configured to permit access to the interior space from the upper section while the lower part of the two-way zipper remains closed and from the lower section while the upper part of the two-way zipper remains closed; and
a wire frame configured to be positioned in the interior space and to support the net enclosure, the wire frame including a plurality of vertical members and a plurality of horizontal members.

19. The indoor/outdoor nature net assembly of claim 18, wherein the upper section of the net enclosure includes at least one upper section aperture configured to receive an upper end of at least one of the vertical members of the wire frame, the indoor/outdoor nature net assembly further including at least one lock configured to attach to the vertical member(s) to prevent the upper section aperture(s) from inadvertently slipping off the vertical member(s).

20. The indoor/outdoor nature net assembly of claim 18, further comprising a cap configured to cover the upper section of the net enclosure.

21. The indoor/outdoor nature net assembly of claim 18, further comprising a sock configured to be filled with sand and wrapped around a lower perimeter of the lower section.

22. The indoor/outdoor nature net assembly of claim 18, further comprising:
a plate that includes a plate aperture configured to receive an upper end of at least one of the vertical members of the wire frame; and
a cover enclosure configured to enclose the net enclosure and the wire frame.
